(19) United States
(12) Patent Application Publication
(10) Pub. No.: US 2024/0315843 A1
(43) Pub. Date: Sep. 26, 2024

(54) PROSTHETIC HEART VALVE DELIVERY ASSEMBLY

(71) Applicant: Medtronic, Inc., Minneapolis, MN (US)

(72) Inventors: Luke A. CLARKE, Galway (IE); Constantin F. CIOBANU, Lackagh (IE); Patricia MCAFEE, Galway (IE); Fionnuala MORELLI, Belfast (GB); Timothy Desmond FARRELL, Tralee (IE); Colm CALLAGY, Loughrea (IE); Chris MORAN, Mayo (IE)

(73) Assignee: Medtronic, Inc., Minneapolis, MN (US)

(21) Appl. No.: 18/582,940

(22) Filed: Feb. 21, 2024

Related U.S. Application Data
(60) Provisional application No. 63/454,450, filed on Mar. 24, 2023.

(51) Int. Cl. A61F 2/24

(52) U.S. Cl. CPC A61F 2/2436

(57) ABSTRACT

A prosthetic valve delivery assembly includes a dilator including a first tapered region extending along a dilator axis. The first tapered region includes a tapered shape with a first diameter at a distal end and a second diameter at a first central end. The second diameter is greater than the first diameter. A central region includes the second diameter that is substantially constant along a central length of the central region. A second tapered region includes a tapered shape with the second diameter at the second central end and a third diameter at a third central end. The third diameter is less than the second diameter. A proximal shaft region extends from the third central end. The proximal shaft region includes the third diameter such that a difference between the second diameter and the third diameter is within a French gauge range from about 3 Fr to about 5 Fr.

### CROSS-REFERENCE TO RELATED APPLICATIONS

This application claims the benefit of U.S. Provisional Patent Application Ser. No. 63/454,450, filed Mar. 24, 2023, the entire content of which is incorporated herein by reference.

### FIELD

The present disclosure relates generally to a prosthetic heart valve delivery assembly and, more particularly, to a prosthetic heart valve delivery assembly comprising a dilator with a non-constant diameter.

### BACKGROUND

It is known to provide a prosthetic heart valve assembly for implanting a heart valve prosthesis within a target site of the vasculature of a patient. It is further known to use a dilator and a sheath as part of the implant procedure. However, insertion of the dilator and the sheath within the vasculature can be difficult.

### SUMMARY

The following presents a simplified summary of the disclosure to provide a basic understanding of some aspects described in the detailed description.

In aspects, a prosthetic valve delivery assembly comprises a dilator extending along a dilator axis between a proximal end and a distal end. The dilator comprises a first tapered region extending along the dilator axis between the distal end and a first central end. The first tapered region comprises a tapered shape with a first diameter at the distal end and a second diameter at the first central end. The second diameter is greater than the first diameter. The dilator comprises a central region coaxial with the first tapered region and attached to the first central end. The central region comprises the second diameter that is substantially constant along a central length of the central region. The dilator comprises a second tapered region extending along the dilator axis between a second central end and a third central end. The second central end is attached to the central region. The second tapered region comprises a tapered shape with the second diameter at the second central end and a third diameter at the third central end. The third diameter is less than the second diameter. The dilator comprises a proximal shaft region extending from and in contact with the third central end. The proximal shaft region comprises the third diameter such that a difference between the second diameter and the third diameter is within a French gauge range from about 3 Fr to about 5 Fr.

In aspects, the central region comprises a central length that is within a range from about 1 mm to about 150 mm.

In aspects, the tapered shape of the second tapered region comprises a taper angle that is within a range from about 1 degree to about 30 degrees.

In aspects, the second diameter is less than about 1.5 times a vessel diameter of the vessel.

In aspects, a handle is attached adjacent to the proximal end of the dilator. The handle is axially fixed relative to the dilator and comprises a non-constant cross-sectional size along a length of the handle.

In aspects, the dilator comprises at least one lumen extending axially through the dilator.

In aspects, the at least one lumen comprises a first lumen, a second lumen, and a third lumen. The first lumen is laterally offset from, and between, the second lumen and the third lumen.

In aspects, the dilator comprises a plurality of materials.

In aspects, a prosthetic valve delivery assembly comprises a sheath comprising a wall surrounding an elongated chamber. The sheath is configured to be received within a vessel. The prosthetic valve delivery assembly comprises a dilator configured to be received within the chamber and extending along a dilator axis between a proximal end and a distal end. The dilator comprises a first tapered region extending along the dilator axis between the distal end and a first central end. The first tapered region comprises a tapered shape with a first diameter at the distal end and a second diameter at the first central end. The second diameter is greater than the first diameter. The dilator comprises a central region coaxial with the first tapered region and attached to the first central end. The central region comprises the second diameter that is less than about 1.5 times a vessel diameter of the vessel. The central region comprises a central length that is within a range from about 1 mm to about 150 mm. The dilator comprises a second tapered region extending along the dilator axis between a second central end and a third central end. The second central end is attached to the central region. The second tapered region comprises a tapered shape with the second diameter at the second central end and a third diameter at the third central end. The third diameter is less than the second diameter. The tapered shape of the second tapered region comprises a taper angle that is within a range from about 1 degree to about 30 degrees. The dilator comprises a proximal shaft region extending from and in contact with the third central end. The proximal shaft region comprises the third diameter. The third diameter is within a range from about 60% to about 90% of the second diameter.

In aspects, a diameter of the central region is non-constant along the central length of the central region, and the second diameter is a maximum diameter of the central region.

In aspects, the central region comprises a first axial location and a second axial location comprising the second diameter. The central region comprises a third axial location positioned between the first axial location and the second axial location and comprising a diameter less than the second diameter.

In aspects, a cross-sectional size of the central region is non-constant about a circumferential perimeter of the central region.

In aspects, the second diameter is constant along the central length of the central region.

In aspects, the central region comprises a material that is different than a material of the proximal shaft region.

In aspects, methods of expanding a sheath comprise positioning a sheath within a vessel. The sheath comprises a wall surrounding an elongated chamber. Methods comprise inserting a dilator within the chamber. The dilator extends along a dilator axis between a proximal end and a distal end. The dilator comprises a first tapered region with an increasing diameter from the distal end, a central region coaxial with and in contact with the first tapered region and comprising a substantially constant diameter, and a second tapered region extending from and in contact with the central region. The second tapered region comprises a tapered shape with a decreasing diameter from the central region. Methods comprise radially expanding the sheath by contacting the wall with the central region.

In aspects, the central region comprises a central length that is within a range from about 1 mm to about 150 mm.

In aspects, methods further comprise moving the dilator axially by applying a force to a handle that is attached adjacent to the proximal end of the dilator. The handle is fixed relative to the dilator and comprises a non-constant cross-sectional size along a length of the handle.

In aspects, methods further comprise receiving a first guidewire within a first lumen that extends axially through the dilator.

In aspects, methods further comprise receiving a second guidewire within a second lumen that extends axially through the dilator, and a third guidewire within a third lumen that extends axially through the dilator. The first lumen is laterally offset from, and between, the second lumen and the third lumen.

In aspects, methods further comprise radially expanding the vessel by contacting a vessel wall of the vessel with the central region.

Additional features and advantages of the aspects disclosed herein will be set forth in the detailed description that follows, and in part will be clear to those skilled in the art from that description or recognized by practicing the aspects described herein, including the detailed description which follows, the claims, as well as the appended drawings. It is to be understood that both the foregoing general description and the following detailed description present aspects intended to provide an overview or framework for understanding the nature and character of the aspects disclosed herein. The accompanying drawings are included to provide further understanding and are incorporated into and constitute a part of this specification. The drawings illustrate various aspects of the disclosure, and together with the description explain the principles and operations thereof.

### BRIEF DESCRIPTION OF THE DRAWINGS

These and other features, aspects and advantages are better understood when the following detailed description is read with reference to the accompanying drawings, in which:

FIG. 1 schematically illustrates example aspects of a transcatheter heart valve prosthesis in accordance with aspects of the disclosure;

FIG. 2 illustrates a top-down view of the transcatheter heart valve prosthesis in accordance with aspects of the disclosure;

FIG. 3 illustrates a side view of a delivery assembly for delivering the transcatheter heart valve prosthesis in accordance with aspects of the disclosure;

FIG. 4 illustrates a side view of the delivery assembly for delivering the transcatheter heart valve prosthesis in accordance with aspects of the disclosure;

FIG. 5 illustrates an introducer sheath in accordance with aspects of the disclosure;

FIG. 6 illustrates an introducer sheath in accordance with aspects of the disclosure;

FIG. 7 schematically illustrates a side view of a dilator and a sheath in accordance with aspects of the disclosure;

FIG. 8 illustrates a perspective view of an end of the dilator in accordance with aspects of the disclosure;

FIG. 9 schematically illustrates a side view of a handle attached to the dilator in accordance with aspects of the disclosure;

FIG. 10 schematically illustrates a side view of the dilator and the sheath positioned in a vessel in accordance with aspects of the disclosure;

FIG. 11 schematically illustrates a side view of the dilator positioned in a vessel in accordance with aspects of the disclosure

FIG. 12 illustrates additional aspects of a dilator in accordance with aspects of the disclosure;

FIG. 13 illustrates additional aspects of a dilator in accordance with aspects of the disclosure;

FIG. 14 illustrates additional aspects of a dilator in accordance with aspects of the disclosure;

FIG. 15 illustrates additional aspects of a dilator in accordance with aspects of the disclosure;

FIG. 16 illustrates additional aspects of a dilator in accordance with aspects of the disclosure;

FIG. 17 illustrates a cross-sectional view of the dilator of FIG. 16 along lines 16-16 of FIG. 16 in accordance with aspects of the disclosure

FIG. 18 illustrates additional aspects of a dilator in accordance with aspects of the disclosure; and

FIG. 19 illustrates additional aspects of a dilator in accordance with aspects of the disclosure.

### DETAILED DESCRIPTION

Aspects will now be described more fully hereinafter with reference to the accompanying drawings in which example aspects are shown. Whenever possible, the same reference numerals are used throughout the drawings to refer to the same or like parts. However, this disclosure may be embodied in many different forms and should not be construed as limited to the aspects set forth herein.

As used herein, the term “about” means that amounts, sizes, formulations, parameters, and other quantities and characteristics are not, and need not be, exact, but may be approximate and/or larger or smaller, as desired, reflecting tolerances, conversion factors, rounding off, measurement error and the like, and other factors known to those of skill in the art.

Ranges can be expressed herein as from “about” one value, and/or to “about” another value. When such a range is expressed, aspects include from the one value to the other value. Similarly, when values are expressed as approximations by use of the antecedent “about,” it will be understood that the value forms another aspect. It will be further understood that the endpoints of each of the ranges are significant both in relation to the other endpoint, and independently of the other endpoint.

Directional terms as used herein—for example up, down, right, left, front, back, top, bottom, upper, lower, etc.—are made only with reference to the figures as drawn and are not intended to imply absolute orientation.

Unless otherwise expressly stated, it is in no way intended that any methods set forth herein be construed as requiring that its steps be performed in a specific order, nor that with any apparatus, specific orientations be required. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred in any respect. This holds for any possible non-express basis for interpretation, including matters of logic relative to arrangement of steps, operational flow, order of components, or orientation of components; plain meaning derived from grammatical organization or punctuation, and; the number or type of aspects described in the specification.

As used herein, the singular forms “a,” “an,” and “the” include plural references unless the context clearly dictates otherwise. Thus, for example, reference to “a” component includes aspects having two or more such components, unless the context clearly indicates otherwise.

The word “exemplary,” “example,” or various forms thereof are used herein to mean serving as an example, instance, or illustration. Any aspect or design described herein as “exemplary” or as an “example” should not be construed as preferred or advantageous over other aspects or designs. Furthermore, examples are provided solely for purposes of clarity and understanding and are not meant to limit or restrict the disclosed subject matter or relevant portions of this disclosure in any manner. It can be appreciated that a myriad of additional or alternate examples of varying scope could have been presented but have been omitted for purposes of brevity.

As used herein, the terms “comprising,” “including,” and variations thereof shall be construed as synonymous and open-ended, unless otherwise indicated. A list of elements following the transitional phrases comprising or including is a non-exclusive list, such that elements in addition to those specifically recited in the list may also be present.

The terms “substantial,” “substantially,” and variations thereof as used herein are intended to represent that a described feature is equal or approximately equal to a value or description. For example, a “substantially planar” surface is intended to denote a surface that is planar or approximately planar. Moreover, “substantially” is intended to denote that two values are equal or approximately equal. The term “substantially” may denote values within about 10% of each other, for example, within about 5% of each other, or within about 2% of each other.

Modifications may be made to the instant disclosure without departing from the scope or spirit of the claimed subject matter. Unless specified otherwise, “first,” “second,” or the like are not intended to imply a temporal aspect, a spatial aspect, an ordering, etc. Rather, such terms are merely used as identifiers, names, etc. for features, elements, items, etc. For example, a first end and a second end generally correspond to end A and end B or two different ends.

Unless otherwise indicated, the terms “distal” and “proximal” are used in the following description with respect to a position or direction relative to the treating clinician. “Distal” and “distally” are positions distant from or in a direction away from the clinician, and “proximal” and “proximally” are positions near or in a direction toward the clinician.

In addition, the term “self-expanding” may be used in the following description with reference to one or more valve or stent structures of the prostheses hereof and is intended to convey that the structures are shaped or formed from a material that can be provided with a mechanical memory to return the structure from a compressed or constricted delivery configuration to an expanded deployed configuration or vice versa. Non-exhaustive exemplary self-expanding materials include stainless steel, a pseudo-clastic metal such as a nickel titanium alloy or nitinol, various polymers, or a so-called super alloy, which may have a base metal of nickel, cobalt, chromium, or other metal. Mechanical memory may be imparted to a wire or stent structure by thermal treatment to achieve a spring temper in stainless steel, for example, or to set a shape memory in a susceptible metal alloy, such as nitinol. Various polymers that can be made to have shape memory characteristics may also be suitable for use in aspects hereof to include polymers such as polynorborene, trans-polyisoprene, styrene-butadiene, and polyurethane. As well poly L-D lactic copolymer, oligo caprylactone copolymer and poly cyclo-octine can be used separately or in conjunction with other shape memory polymers.

Diseases associated with heart valves, such as those caused by damage or a defect, can include stenosis and valvular insufficiency or regurgitation. For example, valvular stenosis causes the valve to become narrowed and hardened which can prevent blood flow to a downstream heart chamber from occurring at the proper flow rate and may cause the heart to work harder to pump the blood through the diseased valve. Valvular insufficiency or regurgitation occurs when the valve does not close completely, allowing blood to flow backwards, thereby causing the heart to be less efficient. A diseased or damaged valve, which can be congenital, age-related, drug-induced, or in some instances, caused by infection, can result in an enlarged, thickened heart that loses elasticity and efficiency. Some symptoms of heart valve diseases can include weakness, shortness of breath, dizziness, fainting, palpitations, anemia and edema, and blood clots which can increase the likelihood of stroke or pulmonary embolism. Symptoms can often be severe enough to be debilitating and/or life threatening.

Heart valve prostheses have been developed for repair and replacement of diseased and/or damaged heart valves. Such heart valve prostheses can be percutaneously delivered and deployed at the site of the diseased heart valve through catheter-based delivery systems. Such heart valve prostheses generally include a frame or stent and a prosthetic valve mounted within the frame. Such heart valve prostheses are delivered in a radially compressed or crimped configuration so that the heart valve prosthesis can be advanced through the patient's vasculature. Once positioned at the treatment site, the heart valve prosthesis is expanded to engage tissue at the diseased heart valve region to, for instance, hold the heart valve prosthesis in position.

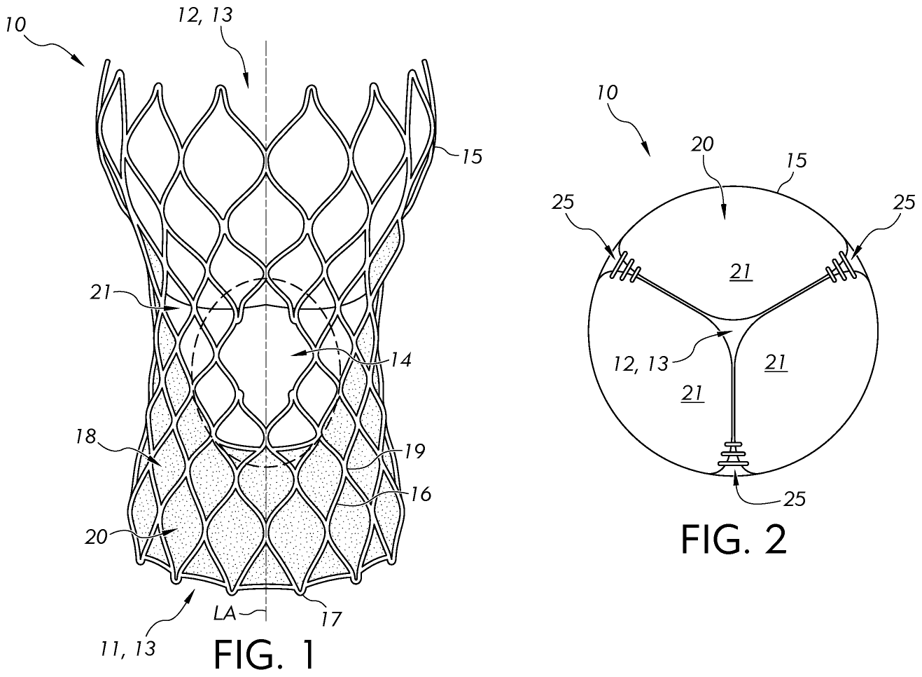

FIGS. 1 and 2 illustrate an example transcatheter heart valve prosthesis 10. The delivery assemblies described herein may be used with the transcatheter heart valve prosthesis 10 and/or other transcatheter heart valve prostheses. The transcatheter heart valve prosthesis 10 is illustrated to facilitate description of the disclosure. The following description of the transcatheter heart valve prosthesis 10 is merely exemplary in nature and is not intended to limit the invention or the application and uses of the invention.

FIGS. 1 and 2 illustrate a side view and a top (outflow end) view, respectively, of the transcatheter heart valve prosthesis 10. The transcatheter heart valve prosthesis 10 includes a radially-expandable frame or stent 15 and a prosthetic valve 20. The frame 15 of the transcatheter heart valve prosthesis 10 supports the prosthetic valve 20 within an interior of the frame 15. In the example transcatheter heart valve prosthesis 10 shown in FIGS. 1 and 2, the frame 15 is self-expandable. However, this is not meant to be limiting, and the frame 15 can be balloon-expandable or mechanically expandable in other embodiments.

The prosthetic valve 20 includes at least one leaflet 21 disposed within and secured to the frame 15. In the embodiment shown in FIGS. 1 and 2, the prosthetic valve 20 includes exactly three leaflets 21, as shown in FIG. 2. However, this is not meant to be limiting, as the prosthetic valve 20 may include more or fewer leaflets 21. The valve leaflets 21 open and close to regulate flow through the transcatheter heart valve prosthesis 10.

As shown in FIG. 1, the transcatheter heart valve prosthesis 10 includes an inflow end 11 and an outflow end 12. The prosthetic leaflets 21 are attached to the frame 15 at commissures 25 such that when pressure at the inflow end 11 exceeds pressure at the outflow end 12, the prosthetic leaflets 21 open to allow blood flow through the heart valve prosthesis 10 from the inflow end 11 to the outflow end 12. When the pressure at the outflow end 12 exceeds pressure at the inflow end 11, the prosthetic leaflets 21 close to prevent blood flow from the outflow end 12 to the inflow end 11. Accordingly, the at least one leaflet (e.g., the prosthetic leaflets 21) can be attached to the plurality of struts 16, for example, by being directly attached to the plurality of struts 16 at the commissures 25, or by being indirectly attached to the plurality of struts 16, for example, by being attached to a skirt, a commissure bracket, or other structure (e.g., mechanical actuator) that is attached to the plurality of struts 16.

The frame 15 of the transcatheter heart valve prosthesis 10 further includes a plurality of struts 16 that are arranged to form a plurality of openings or cells 18 arranged circumferentially around a longitudinal axis LA of the transcatheter heart valve prosthesis 10 and longitudinally to form a tubular structure defining a central lumen 13 of the transcatheter heart valve prosthesis 10. For example, the frame 15 can extend along the longitudinal axis LA between the inflow end 11 and the outflow end 12. The frame 15 is configured to secure the prosthetic valve 20 within the central lumen 13 of the frame 15 and to secure the transcatheter heart valve prosthesis 10 in place in the vasculature of the patient. The struts 16 are defined herein as the elongated wire segments of the frame 15. Struts 16 come together to form crowns 17 or nodes 19, as can be seen in FIG. 1. The frame 15 of the heart valve prosthesis 10 includes a plurality of cells 18 defined as the spaces between the plurality of crowns 17, the plurality of nodes 19, and the plurality of struts 16. The frame 15, and, thus, the plurality of struts 16, can be adjustable between a radially-collapsed position and a radially-expanded position.

In the example embodiment shown in FIG. 1, the plurality of cells 18 may be diamond-shaped. In the example embodiment shown, the plurality of cells include a plurality of first cells 18 and access cells 14. In particular, the access cells are larger than the first cells 18 and can provide access to one or more coronary arteries when the transcatheter heart valve prosthesis 10 is implanted in the patient. In the embodiment shown, there are exactly three access cells 14. However, this is not meant to be limiting, as the frame 15 of the transcatheter heart valve prosthesis 10 can include more, fewer, or no access cells 14. The access cells 14 each have an enlarged area relative or compared to the first cells 18, as can be seen in FIG. 1. Further, the access cells 14 may be located in other locations than the locations shown in FIG. 1. Although not shown, in some embodiments the transcatheter heart valve prosthesis 10 may include an outer skirt extending circumferentially around an outer circumference of the stent 15 at or near the inflow end 11 to prevent paravalvular leakage of blood around the outside of the transcatheter heart valve prosthesis 10 once implanted in the patient.

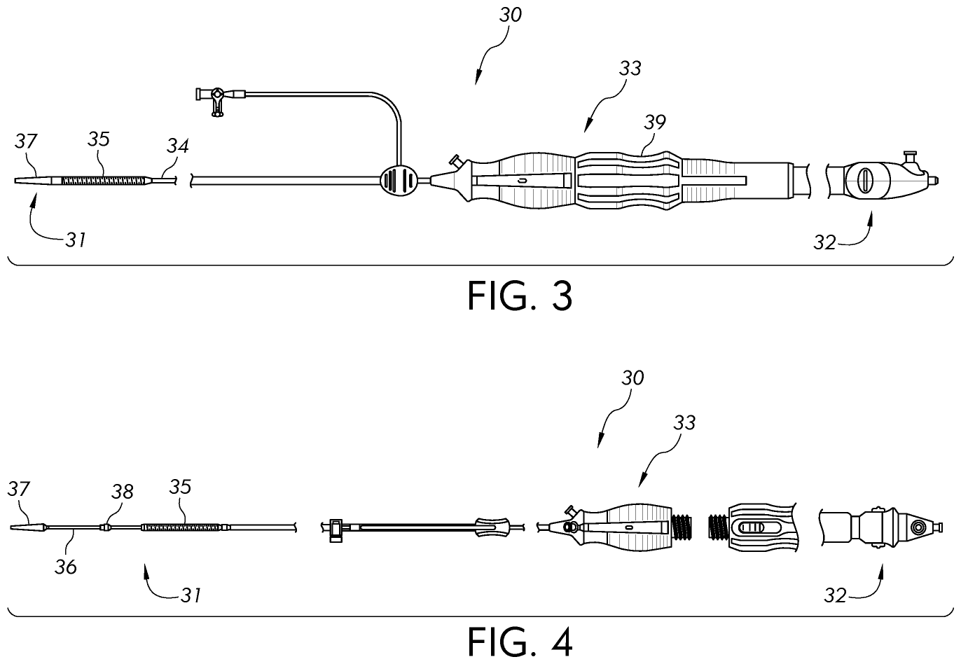

FIGS. 3 and 4 show schematically side views of a delivery assembly 30 for delivering and deploying a transcatheter heart valve prosthesis (e.g., transcatheter heart valve prosthesis 10) according to embodiments hereof. One skilled in the art will realize that FIGS. 3 and 4 illustrate one example of a delivery assembly 30 and that components illustrated in FIGS. 3 and 4 may be removed and/or additional components may be added. The delivery assembly 30 includes a distal end 31, a proximal end 32, and a handle 33. The handle 33 enables a physician to manipulate a distal portion of the delivery assembly 30 and includes actuators for moving parts of the delivery assembly 30 relative to other parts. In the delivery assembly 30, an outer shaft 34 is coupled to an actuator 39 of the handle 33 for moving the outer shaft 34 relative to an inner shaft 36.

A distal portion of the outer shaft 34, referred to as a capsule 35, is configured to surround a transcatheter heart valve prosthesis (e.g., transcatheter heart valve prosthesis 10) during delivery to the treatment site (e.g., a native heart valve) and is retracted from the transcatheter heart valve prosthesis to expose the transcatheter heart valve prosthesis such that it self-expands. The inner shaft 36 is coupled to the handle 33 and movement of the handle 33 translates to movement of the inner shaft 36 and a distal tip or nosecone 37 coupled to a distal end of the inner shaft 36. The inner shaft 36 and distal tip or nosecone 37 may also be translated relative to the outer shaft 34 and the handle 33 via a tip retractor. In the embodiment shown, the inner shaft 36 includes a retainer or spindle 38 for receiving the paddles of the transcatheter heart valve prosthesis 10.

When the actuator 39 is actuated, the actuator 39 moves the outer shaft 34 and the capsule 35 relative to the inner shaft 36, as shown in FIG. 4. As known to those skilled in the art, when the delivery assembly 30 is in position such that the transcatheter heart valve prosthesis 10 is at the desired position at the treatment site in the patient's vasculature, the actuator 39 is actuated to move the capsule 35 relative to the inner shaft 36 and the transcatheter heart valve prosthesis 10 disposed between the inner shaft 36 and the capsule 35, thereby enabling the transcatheter heart valve prosthesis 10 to deploy via self-expansion at the treatment site and release from the retainer 38, as shown in FIG. 4 (without showing the transcatheter heart valve prosthesis 10).

Minimally invasive percutaneous interventional procedures, including endovascular procedures, require access to the venous or arterial system. In general, it is desirable to make the smallest incision point with the shortest tissue contact time when entering the body. Small incisions and short tissue contact time generally lead to improved patient outcomes, less complications, and less trauma to the vessels or organs being accessed, as well as less trauma to the skin and tissue through which the access point is created. Access is required for various medical procedures that deliver or implant structural elements (such as heart valves, heart valve repair devices, occluders, grafts, electrical stimulators, leads, etc.) percutaneously. Some procedures employ relatively large devices that require relatively large sheaths to deliver the devices to the intended site within the body. With such procedures, access site trauma can occur, often resulting in vessel damage, excessive bleeding, increased case time, increased risk of infection, and increased hospitalization time. To reduce access trauma, physicians try to use the smallest devices possible and place the smallest sheath size. This can be problematic, however, if during the procedure the physician discovers a larger device is needed. This leads to a need to upsize the sheath, which is a lengthy procedure and leads to increased risk to the patient. Expandable sheaths can be expanded within the body and thus do not require removal to upsize.

Expandable sheath designs may be regionally or locally expansive to selectively and temporarily expand when the device is passing through a region of the sheath and to retract or recover when the device is not passing or has already passed through the sheath. Embodiments disclosed herein may be employed with an expandable introducer sheath that may solve these and other issues that contribute to vascular trauma. The expandable introducer sheath disclosed herein is described with respect to percutaneous access for transcatheter heart valve repair or replacement, and it should be understood that one or more features of the expandable introducer sheath may be employed alone or in combination for other medical procedures requiring percutaneous access, including but not limited to placement of stents, angioplasty, removal of arterial or venous calcification, and pre-dilatation or post-dilatation.

Various embodiments disclosed herein may include an introducer sheath that has a selectively expandable diameter to allow for the passage of a relatively larger device therethrough and further is configured to return to its original diameter upon passage of the device. The various embodiments may reduce damage to surrounding tissues by reducing contact with those tissues and by eliminating the need to exchange sheaths of different sizes. As a result, in comparison to known sheaths, these embodiments can reduce procedure time, vascular trauma, bleeding, and the resulting risk of infection and other complications. However, it should be understood that the present disclosure is not limited for use with an expandable introducer sheath. Rather, one or more features of the present disclosure can be employed either alone or in combination without an introducer sheath, with a non-expandable introducer sheath, or with an expandable introducer sheath. Likewise, if employed, the introducer sheath may be an integrated introducer sheath (e.g., an introducer sheath integrated with a delivery assembly) or a non-integrated introducer sheath (e.g., an introducer sheath separate from the delivery assembly but provided for use with the delivery assembly).

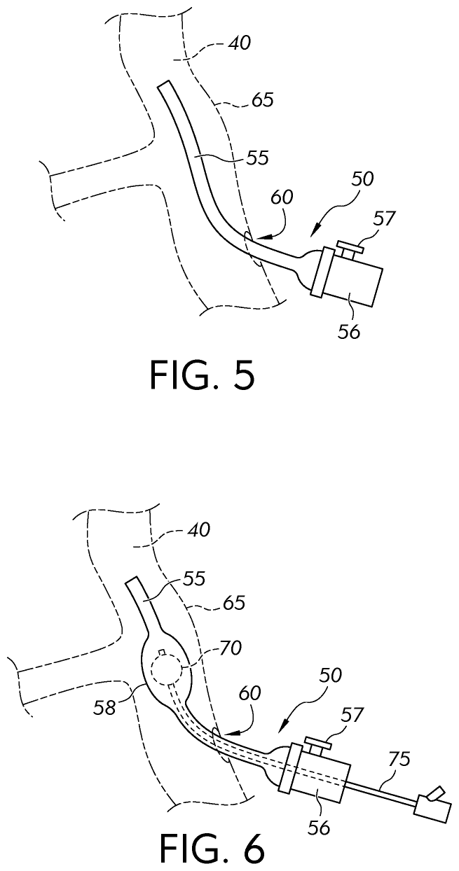

FIGS. 5 and 6 depict one embodiment of an introducer sheath 50 positioned through an incision 60 in the skin 65 of a patient and into a vessel 40 of a patient. The sheath 50 has a tubular shaft 55 and a proximal hub 56 with a hemostatic seal and a luer lock 57. FIG. 5 shows the sheath 50 positioned in the vessel 40 in its normal, unexpanded state, while FIG. 6 shows the sheath 50 positioned in the vessel 40 with a delivery device 75 delivering another device 70 that is being advanced through the sheath 50 such that the tubular shaft 55 expands or deforms at the location where the device 70 is passing through. The shaft 55 expands at expanded region 58 when the device 70 passes through and then retracts or recovers to its original diameter after the device 70 moves past or is removed from the shaft 55. Thus, the tubular shaft 55 is configured to be expandable and retractable.

In certain embodiments, the expandability of the shaft 55 (and any shaft described according to any embodiment set forth herein) is achieved via the elasticity of the shaft 55, which can result in the shaft 55 being either self-expandable or self-expanding or mechanically expandable or mechanically expanding. For purposes of this application, self-expandable means that the shaft 55 is configured to expand to a predetermined or nominal diameter automatically (without any type of actuation, mechanical or otherwise). Further, for purposes of this application, mechanically expandable means that the shaft 55 is configured to expand when a positionable medical device is positioned through the shaft 55. That is, the device itself that is being passed through the shaft 55 causes the expansion of the shaft 55, as depicted in FIG. 6. Alternatively, the expandable characteristics of the shaft 55 can be caused by something other than elasticity.

After passage of the device, the shaft 55 is configured to be contractable, retractable, or recoverable to its original, unexpanded state as depicted in FIG. 5. The retractability can be, in certain embodiments, achieved by the elasticity of the shaft 55, which can result in the shaft 55 being either self-retractable or self-retracting, self-recoverable, or self-contractable, or mechanically retractable or mechanically retracting, mechanically recoverable, or mechanically contractable. For purposes of this application, self-retractable means that the shaft 55 is configured to retract to a predetermined or nominal diameter automatically (without any type of actuation, mechanical or otherwise). Further, for purposes of this application, mechanically retractable means that the shaft 55 is configured to retract when a device or component is used to cause the shaft 55 to retract or recover. Alternatively, the retractable characteristics of the shaft 55 can be caused by something other than elasticity.

For purposes of this application, any device that can be positioned through an introducer sheath according to any embodiment disclosed or contemplated herein can be referred to as a positionable medical device or insertable medical device. Such devices include guidewires, dilators, delivery devices (for delivery and/or placement of structural elements such as heart valves, heart valve repair devices, occluders, grafts, electrical stimulators, leads, etc.), guide catheters, guiding sheaths, diagnostic catheters, stent delivery systems, balloon catheters, and other known vascular devices. Other devices can include non-vascular devices such as scopes and other common surgical instruments. Further, the introducer sheath is configured to receive tissues or organs. Thus, as one non-limiting example, the introducer sheath 50 is described as being an expandable introducer sheath 50 for introduction of a delivery assembly 30 including a transcatheter heart valve prosthesis 10.

As discussed more fully below, dilators and introducer sheaths (e.g., introducer sheath 50) may be provided as a pair of components that can be included in a vascular introducer set. An introducer set may contain devices used to access blood vessels (e.g., vessel 40) for the insertion of vascular catheters (e.g., delivery assembly 30). For example, after a needle is inserted through the skin 65 at incision 60 and into the blood vessel 40, a dilator and sheath 50 are passed together into the blood vessel 40. The tapered tip of the dilator acts to stretch the opening 60 in the skin 65 and the blood vessel 40 to allow for the insertion of the introducer sheath 50. The dilator may also be employed to dilate the introducer sheath 50 within the vessel 40. The dilator is then removed, leaving only the introducer sheath 50 inserted into the blood vessel 40, providing an access port through which a variety of catheters (e.g., delivery assembly 30) can be inserted.

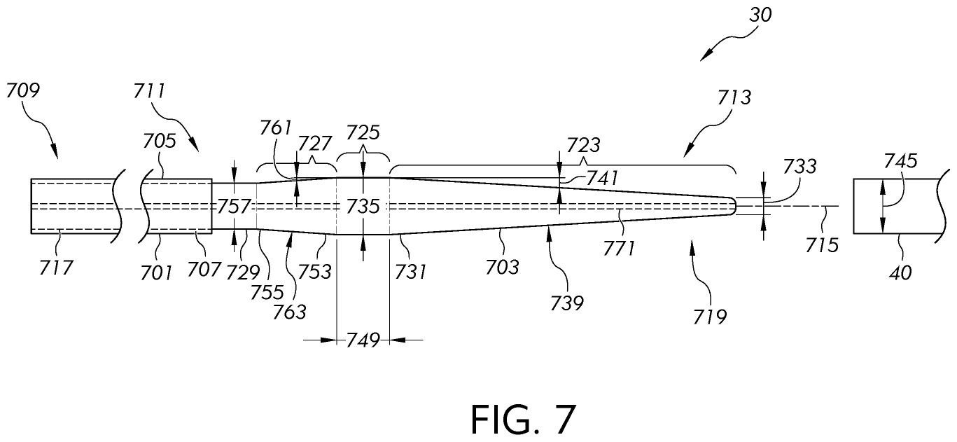

FIG. 7 illustrates a side view of a dilator 703 in accordance with aspects of the disclosure. The dilator 703 can be employed with a sheath 701 (e.g., introducer sheath 50) cither alone or in combination with delivery assembly 30. The dilator 703 can be received within the vessel 40 to dilate the vessel 40. For purposes of illustration, the sheath 701 and the dilator 703 are illustrated as being outside of the vessel 40 in FIG. 7. However, during the process of delivering the valve prosthesis 10, the sheath 701 and the dilator 703 can be positioned within the vessel 40. The sheath 701 comprises a wall 705 surrounding an elongated chamber 707. The sheath 701 can extend between a proximal end 709 and a distal end 711, and, in aspects, the proximal end 709 may be attached to, or in operative association with, the proximal hub 56. In aspects, the sheath 701 is received within the vessel 40 and can be accessed through the proximal hub 56. The dilator 703 can be received within the chamber 707. The dilator 703 can extend along a dilator axis 715 between a proximal end 717 and a distal end 719.

The dilator 703 can comprise a tapered end portion 713 comprising one or more regions, for example, a first tapered region 723, a central region 725, a second tapered region 727, and a proximal shaft region 729. With regard to the first tapered region 723, the first tapered region 723 can extend along the dilator axis 715 between the distal end of the dilator 703 and a first central end 731. The first tapered region 723 can comprise a length (e.g., distance between the distal end 719 and the first central end 731) that may be within a range from about 10 millimeters (“mm”) to about 100 mm. In aspects, the first tapered region 723 can comprise a tapered shape with a first diameter 733 at the distal end 719 and a second diameter 735 at the first central end 731, with the second diameter 735 greater than the first diameter 733. The tapered shape can comprise a decreasing cross-sectional size (e.g., diameter) from the first central end 731 to the distal end 719. In aspects, the first tapered region 723 can comprise a constant tapered shape that decreases in diameter at a constant rate from the first central end 731 to the distal end 719. In aspects, an outer radial surface 739 of the first tapered region 723 can form an angle 741 relative to an axis that is parallel to the dilator axis 715, with the angle 741 in a range from about 1 degree to about 5 degrees, or from about 2 degrees to about 3 degrees, or about 2.5 degrees. The first tapered region 723 can reduce the likelihood of damage to the vessel 40 when the dilator 703 is inserted into, and moves relative to, the vessel 40.

The central region 725 can be coaxial with the first tapered region 723 and may be attached to the first central end 731 of the first tapered region 723. In this way, the central region 725 and the first tapered region 723 can be continuous, such that the first tapered region 723 abuts the central region 725 at the first central end 731. The central region 725 can comprise the second diameter 735, such that the central region 725 can comprise the same cross-sectional size as the first central end 731 of the first tapered region 723. In aspects, the second diameter 735 can be less than about 1.5 times a vessel diameter 745 of the vessel 40, wherein, in aspects, the vessel diameter 745 may comprise the minimum vessel size of the vessel 40 in the track path of the vasculature. For example, in aspects, the vessel 40 may comprise a femoral artery that may comprise a diameter of about 6 mm, such that the dilator 703 may be selected such that the second diameter 735 is less than about 9 mm. In this way, in aspects, the cross-sectional size of the central region 725 may be greater than the cross-sectional size of the vessel 40. In aspects, the second diameter 735 can be less than a diameter of the valve prosthesis 10 when the valve prosthesis 10 is in the radially-compressed or crimped configuration, for example, with the second diameter 735 within a range from about 90% to about 95% of a diameter of the valve prosthesis 10 when the valve prosthesis 10 is in the radially-compressed or crimped configuration. In aspects, the second diameter 735 may be substantially constant along a central length 749 of the central region 725. For example, the central region 725 can comprise the central length 749 that may be within a range from about 1 mm to about 150 mm, or about 5 mm to about 30 mm, or about 10 mm to about 25 mm. In this way, the central region 725 can comprise a cylindrical shape with a substantially constant cross-sectional size (e.g., the second diameter 735) between the first tapered region 723 and the second tapered region 727. The central length 749 of the central region 725 can be selected to expand the sheath 701 and/or the vessel 70 for a long enough length to ensure that dilation has occurred.

The second tapered region 727 can be coaxial with the first tapered region 723 and the central region 725. The second tapered region 727 can extend along the dilator axis 715 between a second central end 753 and a third central end 755, with the second central end 753 attached to the central region 725. In this way, the central region 725 and the second tapered region 727 can be continuous, such that the central region 725 abuts the second tapered region 727 at the second central end 753. Accordingly, the central region 725 can extend between opposing ends, with a distal end attached to the first tapered region 723 and an opposing proximal end attached to the second tapered region 727. In aspects, the second tapered region 727 can comprise a tapered shape with the second diameter 735 at the second central end 753 and a third diameter 757 at the third central end 755. The third diameter 757 may be less than the second diameter 735. In this way, the tapered shape can comprise a decreasing cross-sectional size (e.g., diameter) from the second central end 753 to the third central end 755. In aspects, the second tapered region 727 can comprise a constant tapered shape that decreases in diameter at a constant rate from the second central end 753 to the third central end 755. The second tapered region 727 can comprise a length (e.g., distance between the second central end 753 and the third central end 755) that may be within a range from about 5 mm to about 100 mm. In aspects, a length of the second tapered region 727 may be less than a length of the first tapered region 723 and/or less than the central length 749 of the central region 725. The tapered shape of the second tapered region 727 can comprise a taper angle 761 that is within a range from about 1 degree to about 30 degrees, or from about 1 degree to about 10 degrees, or from about 2 degrees to about 6 degrees. The taper angle 761 can be measured between an outer radial surface 763 of the second tapered region 727 and an axis that is parallel to the dilator axis 715. The taper angle 761 can be selected to ensure a smooth transition between the varying diameters, for example, a smooth transition between the central region 725 and the second tapered region 727 and a smooth transition between the second tapered region 727 and the proximal shaft region 729. Further, by being less than about 30 degrees, the taper angle 761 can be selected to reduce the likelihood of a kink or other sharp twist or bend at a location between the second tapered region 727 and the proximal shaft region 729.

The proximal shaft region 729 can extend from, and may be in contact with, the third central end 755. For example, the proximal shaft region 729 can be attached to the third central end 755 of the second tapered region 727 such that the proximal shaft region 729 and the second tapered region 727 can be continuous, such that the proximal shaft region 729 abuts the second tapered region 727 at the third central end 755. The proximal shaft region 729 can comprise the third diameter 757 that may be within a range from about 60% to about 90% of the second diameter 735. In this way, the proximal shaft region 729 may comprise a cross-sectional size (e.g., third diameter 757) that is less than a cross-sectional size (e.g., second diameter 735) of the central region 725. In aspects, a difference between the second diameter 735 and the third diameter 757 may be within a French gauge range from about 3 Fr (e.g., corresponding to about 1 mm or a circumference of about 3.14 mm) to about 5 Fr (e.g., corresponding to about 1.67 mm or a circumference of about 5.24 mm), or about 4 Fr. In aspects, the third diameter 757 can be within a range from about 65% to about 95% of the second diameter 735. The proximal shaft region 729 can extend between the proximal end 717 of the dilator 703 and the third central end 755 of the second tapered region 727 with the substantially constant third diameter 757 along the length of the proximal shaft region 729. The cross-sectional size difference between the proximal shaft region 729 and the central region 725 can allow for a balance between the flexural rigidity and the axial rigidity of the dilator 703. For example, if the diameter of the proximal shaft region 729 is too small, then the proximal shaft region 729 may not comprise sufficient pushability (e.g., axial rigidity) to expand the sheath 701 and the vessel 40. If the diameter of the proximal shaft region 729 is too large, then the proximal shaft region 729 may be difficult to move the dilator 703 through the vessel 40 due to higher flexural rigidity and contact surface area of the dilator 703.

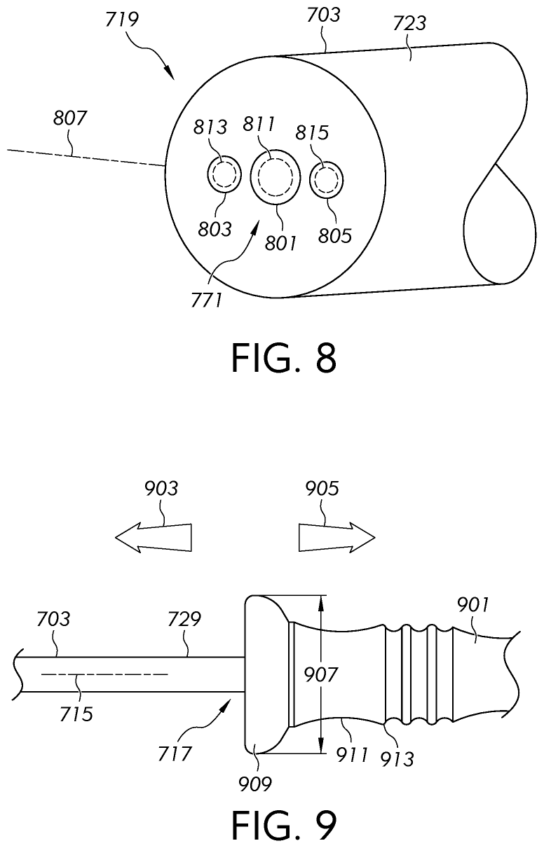

The dilator 703 can comprise at least one lumen 771 extending through the dilator 703 parallel to the dilator axis 715 between the proximal end 717 and the distal end 719. The at least one lumen 771 may be substantially hollow such that at least one guidewire can be received within the at least one lumen 771. In aspects, the regions 723, 725, 727 may be closed in a radial direction and devoid of openings extending in the radial direction such that the regions 723, 725, 727 may circumferentially surround the at least one lumen 771. FIG. 8 illustrates a perspective view of the distal end 719 of the dilator 703. In aspects, the at least one lumen 771 can comprise a plurality of lumen, for example, a first lumen 801, a second lumen 803, and a third lumen 805. The first lumen 801 can be laterally offset from, and between, the second lumen 803 and the third lumen 805. For example, a radial axis 807 can intersect the first lumen 801, the second lumen 803, and the third lumen 805 (e.g., while extending in a radial direction through the dilator 703), with the axis 807 substantially perpendicular to the dilator axis 715 along which the first lumen 801 can extend. In this way, the first lumen 801 may be positioned at a center of the dilator 703, with the second lumen 803 on a first side of the first lumen 801 and the third lumen 805 on an opposing second side of the first lumen 801.

In aspects, each of the lumen 801, 803, 805 can receive a guidewire. For example, the first lumen 801 can receive a first guidewire 811, the second lumen 803 can receive a second guidewire 813, and the third lumen 805 can receive a third guidewire 815. The guidewires 811, 813, 815 can exit the dilator 703 at the distal end 719 and may extend along a length of the dilator 703 to the proximal end 717. The guidewires 811, 813, 815 are illustrated with dashed lines for illustrative purposes and to not obstruct a view of the lumen 801, 803, 805. In aspects, the dilator 703 can move relative to the guidewires 811, 813, 815. In this way, methods of expanding the sheath 701 can comprise receiving the first guidewire 811 within the first lumen 801 that extends axially through the dilator 703. Methods can further comprise receiving the second guidewire 813 within the second lumen 803 that extends axially through the dilator 703, and receiving the third guidewire 815 within the third lumen 805 that extends axially through the dilator 703, with the first lumen 801 laterally offset from, and between, the second lumen 803 and the third lumen 805. While FIG. 8 illustrates the at least one lumen 771 comprising three lumen 801, 803, 805, the dilator 703 is not so limited. Rather, in aspects, the at least one lumen 771 can comprise a single lumen, for example, the first lumen 801, that can receive the first guidewire 811 such that the first lumen 801 and the first guidewire 811 can extend substantially coaxially along the dilator axis 715.

FIG. 9 illustrates a side view of the proximal end 717 of the dilator 703, wherein the dilator 703 can comprise a handle 901 attached to the proximal end 717 of the dilator 703. The handle 901 can be axially-fixed relative to the dilator 703 such that movement of the handle 901 (e.g., along the dilator axis 715) can cause corresponding movement of the dilator 703 along the dilator axis 715. For example, in aspects, the handle 901 can be moved in a first movement direction 903 (e.g., a delivery direction) to move the dilator 703 further into the vessel 40. Due to the handle 901 being axially-fixed, movement of the handle 901 in the first movement direction 903 can cause the dilator 703 to likewise move in the first movement direction 903. Similarly, the handle 901 can be moved in a second movement direction 905 (e.g., a retraction direction) to retract the dilator 703 from the vessel 40. Due to the handle 901 being axially-fixed, movement of the handle 901 in the second movement direction 905 can cause the dilator 703 to likewise move in the second movement direction 905.

In aspects, the handle 901 can comprise a non-constant cross-sectional size along a length of the handle 901. For example, the handle 901 can comprise a first peak portion 909 and a valley portion 911, with the first peak portion 909 comprising a larger cross-sectional size than the valley portion 911. The first peak portion 909 can be closer to the dilator 703 (e.g., the distal end 719, for example) than the valley portion 911. In this way, the first peak portion 909 can be located at an end of the handle 901. In aspects, a second peak portion 913 can be located on an opposite side of the valley portion 911, such that the valley portion 911 is located between the first peak portion 909 and the second peak portion 913. The second peak portion 913 can comprise a larger cross-sectional size than the valley portion 911. Accordingly, a physician can manipulate the handle 901, for example, by holding the handle 901 at the valley portion 911. The physician can move the handle 901, and, thus, the dilator 703, in the first movement direction 903 by applying a force to the first peak portion 909 in the first movement direction 903. Alternatively, the physician can move the handle 901, and, thus, the dilator 703, in the second movement direction 905 by applying a force to the second peak portion 913 in the second movement direction 905. In this way, the non-constant cross-sectional size of the handle 901 can facilitate gripping and manipulating of the handle and allow a movement force to be translated from the handle 901 to the dilator 703. Accordingly, methods can comprise moving the dilator 703 axially by applying a force to the handle 901 that is attached adjacent to the proximal end 717 of the dilator 703, with the handle 901 fixed (e.g., axially fixed) relative to the dilator 703 and comprising a non-constant cross-sectional size along a length of the handle 901.

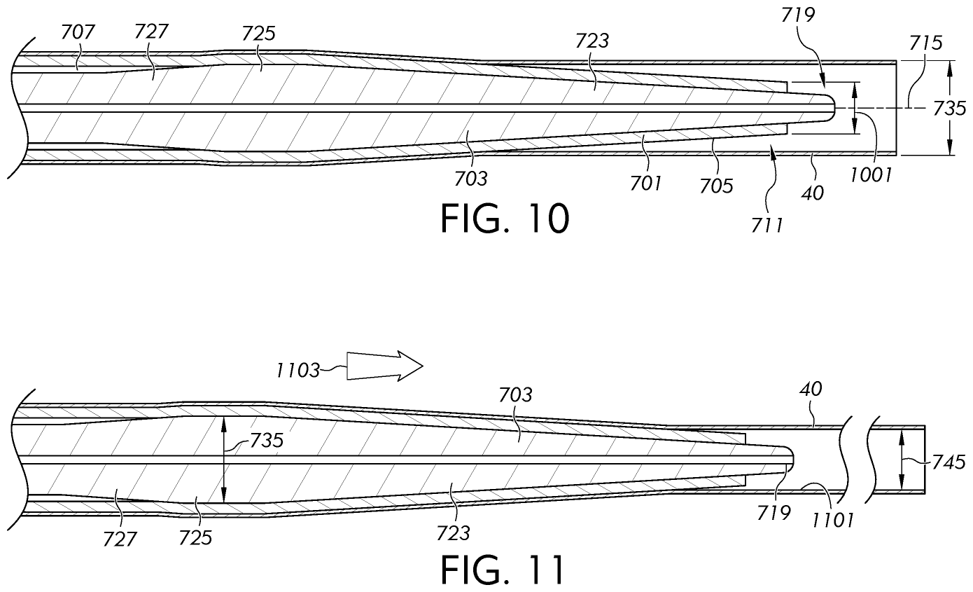

FIG. 10 illustrates the dilator 703 received within the elongated chamber 707 of the sheath 701. Methods can comprise positioning the sheath 701 within the vessel 40, with the sheath 701 comprising the wall 705 surrounding the elongated chamber 707. Methods can further comprise inserting the dilator 703 within the chamber 707. The dilator 703 extends along the dilator axis 715 between the proximal end 717 and the distal end 719, with the dilator 703 comprising the first tapered region 723 with an increasing diameter from the distal end 719, the central region 725 coaxial with and in contact with the first tapered region 723 and comprising a substantially constant diameter, and the second tapered region 727 extending from and in contact with the central region 725. The second tapered region 727 comprises a tapered shape with a decreasing diameter from the central region 725. By receiving the dilator 703 within the sheath 701, methods can comprise radially expanding (e.g., dilating) the sheath 701 by contacting the wall 705 with the central region 725. For example, initially, the sheath 701 can comprise a sheath diameter 1001 that may be less than the second diameter 735 of the central region 725. Upon being inserted into the elongated chamber 707 of the sheath 701, the dilator 703 can cause the sheath 701 to radially expand. For example, the first tapered region 723 and the central region 725 can apply an outward radial force to the wall 705 of the sheath 701, thus causing the sheath 701 to radially expand and increase in diameter. In aspects, when the sheath 701 and the dilator 703 are positioned within the vessel 40, the vessel 40 can likewise radially expand. Accordingly, in the position illustrated in FIG. 10, the central region 725 of the dilator 703 may contact the wall 705 of the sheath 701 and may not contact the vessel 40.

FIG. 11 illustrates the dilator 703 received within the vessel 40 such that the dilator 703 can contact a vessel wall 1101 of the vessel 40. For example, methods can comprise radially expanding the vessel 40 by contacting the vessel wall 1101 with the central region 725. For example, the dilator 703 can be moved in a movement direction 1103 such that the first tapered region 723 can move relative to the vessel wall 1101. As the dilator 703 moves in the movement direction 1103, the first tapered region 723 can apply an outward radial force to the vessel wall 1101. Due to the elongated tapered shape of the first tapered region 723 in which the first tapered region 723 comprises a gradually increasing diameter from the distal end 719, the vessel 40 may be radially-expanded in a gradual manner, thus reducing the risk of damage to the vessel 40. The vessel diameter 745 may continue to increase at least until the vessel wall 1101 contacts the central region 725. The central region 725 is the portion of the dilator 703 comprising a maximum diameter (e.g., the second diameter 735). As such, the central region 725 can cause a maximum radial expansion of the vessel wall 1101 such that the vessel diameter 745 at the portion of the vessel 40 in contact with the central region 725 may be substantially equal to, or slightly larger than, the second diameter 735 of the central region 725. The second tapered region 727 may comprise a decreasing cross-sectional size from the central region 725, such that a portion of the second tapered region 727 may contact the vessel wall 1101. In this way, the central region 725 is the portion of the dilator 703 that exerts a maximum radial force upon the vessel wall 1101, while a lesser or negligible radial force may be exerted by the first tapered region 723, the second tapered region 727, and the proximal shaft region 729. In this way, the total radial force exerted upon the vessel wall 1101 along the length of the dilator 703 may be reduced, since the dilator 703 comprises one region of maximum cross-sectional size (e.g., at the central region 725) while other regions of the dilator 703 comprise a smaller cross-sectional size.

The dilator 703 can comprise multiple functions related to the vessel 40 and the sheath 701. For example, due to the maximum diameter of the dilator 703 at the central region 725, the dilator 703 can dilate and crack calcium in the vessel 40 (e.g., the arteriotomy and iliofemoral vessel, for example). In this way, access through iliofemoral vessels for large bore catheter devices is facilitated. Due to the relative short length of the central region 725, the risk of iliac evulsion is reduced. In addition, when positioned in the sheath 701, the dilator 703 can dilate the expandable sheath 701 to allow for access through the expandable sheath 701 for a large bore catheter device. Due to the size of the second diameter 735 (e.g., which can be about 1 Fr to about 1.5 Fr less than the device (e.g., valve, etc.) that will pass through the expandable sheath 701, the dilator 703 does not induce excess vessel dilation of the vessel 40 since the vessel 40 is dilated below a max dilation that the device (e.g., valve, etc.) may impart when passing through the sheath 701 and/or the vessel 40. The dilator 703 can comprise a material that may reduce contact surface area between the dilator 703 and one or more of the sheath 701 and/or the vessel 40. For example, some or all of the dilator 703 can comprise one or more of a thermoplastic elastomer, a high-density polyethylene, a low-density polyethylene, a thermoplastic polyurethane, or polyamides. In addition, or in the alternative, some or all of the dilator 703 can be coated with a low-friction coating, such as a hydrophilic coating, for example, that can reduce friction and facilitate movement of the dilator 703.

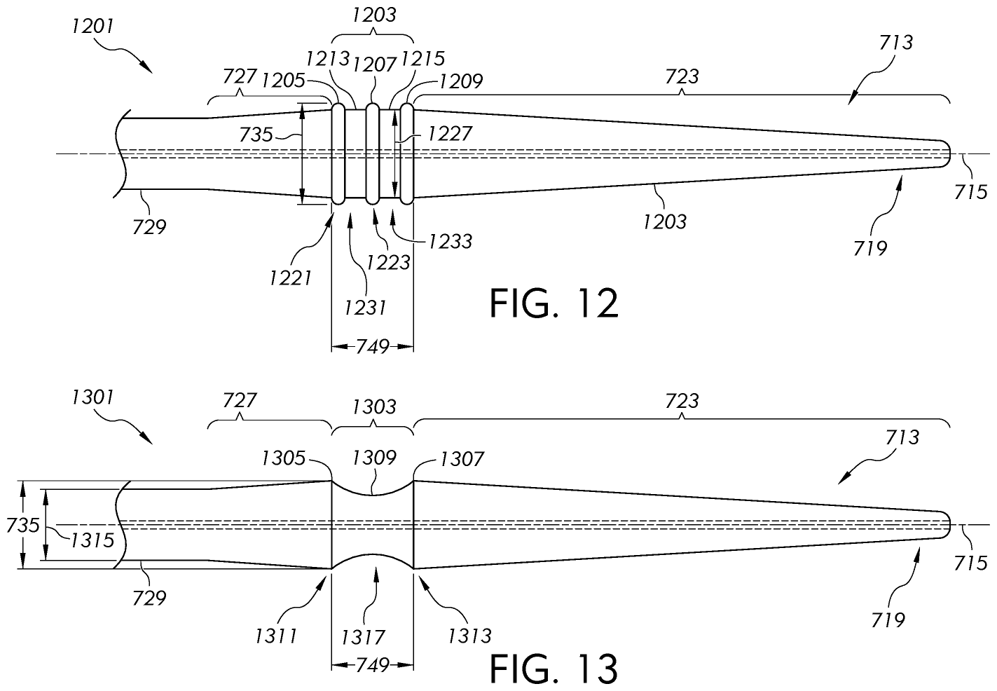

FIG. 12 illustrates an additional embodiment of a dilator 1201 comprising the tapered end portion 713. In aspects, the tapered end portion 713 of the dilator 1201 can comprise some similarities to the tapered end portion 713 of the dilator 703 illustrated in FIGS. 7-11. For example, the first tapered region 723, the second tapered region 727, and the proximal shaft region 729 of the dilator 1201 may be substantially identical to the first tapered region 723, the second tapered region 727, and the proximal shaft region 729 of the dilator 703. The dilator 1201 can comprise a central region 1203 that is positioned between the first tapered region 723 and the second tapered region 727 and comprises some similarities to the central region 725. For example, the central region 1203 can comprise the second diameter 735 that is a maximum diameter of the central region 1203. However, the diameter of the central region 1203 may be non-constant along the central length 749 of the central region 1203, with the central region 1203 comprising one or more peak portions and one or more valley portions.

The central region 1203 can comprise a first peak portion 1205, a second peak portion 1207, and a third peak portion 1209, and a first valley portion 1213 and a second valley portion 1215. The peak portions 1205, 1207, 1209 comprise outcroppings, extensions, projections, protuberances, etc. that extend radially outwardly from the central region 1203 and extend circumferentially around an outer surface of the central region 1203. In aspects, the peak portions 1205, 1207, 1209 can comprise a rounded shape (e.g., rounded along the dilator axis 715) and comprise the second diameter 735. In this way, the peak portions 1205, 1207, 1209 can comprise the maximum diameter of the central region 1203. The peak portions 1205, 1207, 1209 may be substantially identical in size and shape, with the peak portions 1205, 1207, 1209 spaced apart along the dilator axis 715 and located at differing axial locations. For example, the first peak portion 1205 can be located at a first axial location 1221 that is at an end of the central region 1203 adjacent to the second tapered region 727. The second peak portion 1207 can be located at a second axial location 1223 that is near the middle of the central region 1203, with a distance separating the distal end 719 and the second axial location 1223 less than a distance separating the distal end 719 and the first axial location 1221. In aspects, the third peak portion 1209 can be located at an end of the central region 1203 adjacent to the first tapered region 723 such that the first peak portion 1205 and the third peak portion 1209 are at opposing ends of the central region 1203.

The valley portions 1213, 1215 can comprise substantially constant diameters between adjacent peak portions, for example, with the valley portions 1213, 1215 comprising a valley diameter 1227 and extending circumferentially around an outer surface of the central region 1203. In aspects, the valley diameter 1227 may be less than the second diameter 735 such that the valley portions 1213, 1215 comprise a minimum diameter of the central region 1203. The valley portions 1213, 1215 can be spaced apart along the dilator axis 715 and located at differing axial locations. For example, the first valley portion 1213 can be located at a third axial location 1231 that is between the first axial location 1221 and the second axial location 1223. The fourth axial location 1233 can be adjacent to the second axial location 1223 and on an opposite side of the second axial location 1223 from the third axial location 1231. In this way, the first valley portion 1213 may be located axially between the first peak portion 1205 and the second peak portion 1207. The second peak portion 1207 may be located axially between the first valley portion 1213 and the second valley portion 1215. Accordingly, the first axial location 1221 and the second axial location 1223 can comprise the second diameter 735, with the third axial location 1231 positioned between the first axial location 1221 and the second axial location 1223 and comprising the valley diameter 1227 that is less than the second diameter 735. In this way, the dilator 1201 can function substantially identically to the dilator 703 illustrated in FIGS. 7-11, with the central region 1203 causing radial-expansion of the vessel 40 and/or the sheath 701. Due to the central region 1203 comprising the non-constant diameter, the total radial force exerted upon the walls 705, 1101 along the length of the dilator 1201 may be reduced, such as, for example, by being limited to the peak portions 1205, 1207, 1209 that contact the walls 705, 1101.

FIG. 13 illustrates an additional embodiment of a dilator 1301 comprising the tapered end portion 713. In aspects, the first tapered region 723, the second tapered region 727, and the proximal shaft region 729 of the dilator 1301 may be substantially identical to the first tapered region 723, the second tapered region 727, and the proximal shaft region 729 of the dilators 703, 1201. The dilator 1301 can comprise a central region 1303 that is positioned between the first tapered region 723 and the second tapered region 727 and comprises some similarities to the central regions 725, 1203. For example, the central region 1303 can comprise the second diameter 735 that is a maximum diameter of the central region 1303. However, the diameter of the central region 1303 may be non-constant along the central length 749 of the central region 1203, with the central region 1203 comprising one or more peak portions and one or more valley portions.

The central region 1303 can comprise a first peak portion 1305 and a second peak portion 1307, and a valley portion 1309 positioned between the first peak portion 1305 and the second peak portion 1307. The peak portions 1305, 1307 extend circumferentially around an outer surface of the central region 1303. The peak portions 1305, 1307 can comprise the maximum diameter (e.g., the second diameter 735) of the central region 1303. For example, the peak portions 1305, 1307 may be substantially identical in size and shape, with the peak portions 1305, 1307 spaced apart along the dilator axis 715 and located at differing axial locations. For example, the first peak portion 1305 can be located at a first axial location 1311 that is at an end of the central region 1303 adjacent to the second tapered region 727. The second peak portion 1307 can be located at a second axial location 1313 that is near an opposing end of the central region 1303 adjacent to the first tapered region 723, with a distance separating the distal end 719 and the second axial location 1313 less than a distance separating the distal end 719 and the first axial location 1311. In this way, the first peak portion 1305 and the second peak portion 1307 are at opposing ends of the central region 1303.

The valley portion 1309 can comprise a non-constant diameter, for example, by comprising a valley diameter 1315 that is less than the second diameter 735. In aspects, the valley portion 1309 can comprise a rounded shape with a decreasing diameter from the first peak portion 1305 toward a center of the valley portion 1309, and a decreasing diameter from the second peak portion 1307 toward a center of the valley portion 1309. In this way, the valley portion 1309 can comprise a minimum diameter (e.g., the valley diameter 1315) at an axial center of the valley portion 1309 located substantially at a midpoint between the first peak portion 1305 and the second peak portion 1307. The valley portion 1309 is located at a third axial location 1317 that is between the first axial location 1311 and the second axial location 1313. In this way, the valley portion 1309 may be located axially between the first peak portion 1305 and the second peak portion 1307. Accordingly, the first axial location 1311 and the second axial location 1313 can comprise the second diameter 735, with the third axial location 1317 positioned between the first axial location 1311 and the second axial location 1313 and comprising the valley diameter 1315 that is less than the second diameter 735. In this way, the dilator 1301 can function substantially identically to the dilators 703, 1201 illustrated in FIGS. 7-12, with the central region 1303 causing radial-expansion of the vessel 40 and/or the sheath 701. Due to the central region 1303 comprising the non-constant diameter, the total radial force exerted upon the walls 705, 1101 along the length of the dilator 1301 may be reduced, such as, for example, by being limited to the peak portions 1305, 1307 that contact the walls 705, 1101.

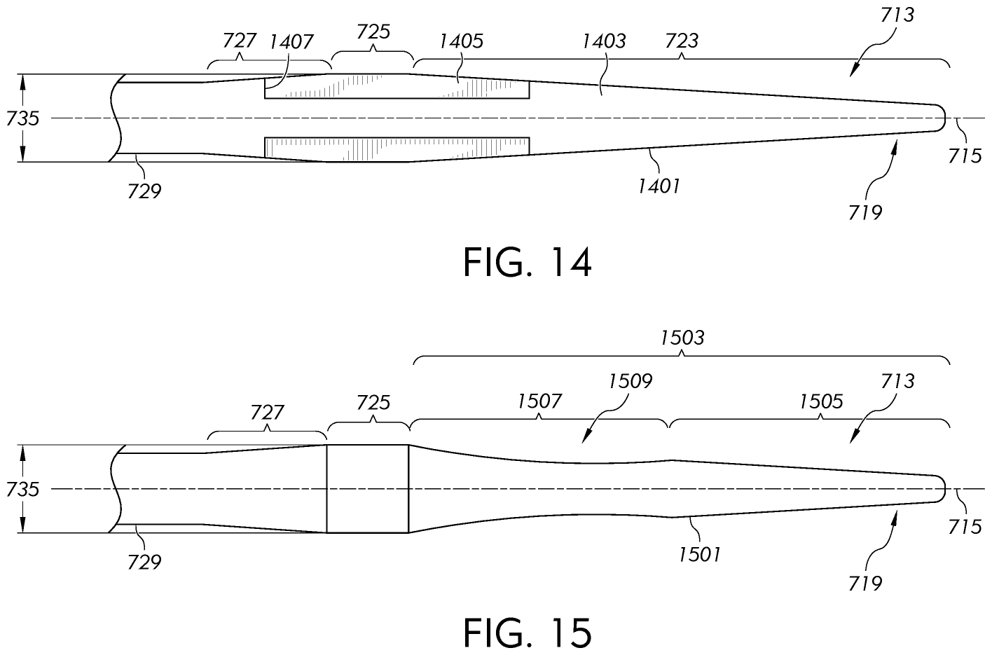

FIG. 14 illustrates an additional embodiment of a dilator 1401 comprising the tapered end portion 713. In aspects, the first tapered region 723, the second tapered region 727, the central region 725, and the proximal shaft region 729 of the dilator 1401 may be substantially identical in shape and function to the first tapered region 723, the second tapered region 727, the central region 725, and the proximal shaft region 729 of the dilator 703. However, the dilator 1401 can comprise a plurality of materials, for example, with the central region 725 comprising a material that is different than a material of the proximal shaft region 729. For example, the dilator 1401 can comprise a first material portion 1403 and a second material portion 1405. The first material portion 1403 can comprise some or all of the first tapered region 723, the second tapered region 727, and the proximal shaft region 729. The second material portion 1405 can comprise some or all of the central region 725, the first tapered region 723, and the second tapered region 727. In aspects, the first material portion 1403 can comprise a different material than the second material portion 1405. In aspects, the second material portion 1405 can be received within a recess 1407 defined within the first material portion 1403. The first material portion 1403 can comprise a portion of the first tapered region 723 and a portion of the second tapered region 727. The second material portion 1405 can comprise the central region 725, a remaining portion of the first tapered region 723, and a remaining portion of the second tapered region 727. In aspects, the first material portion 1403 can comprise a softer and more flexible material than the second material portion 1405. In this way, the first material portion 1403 can bend and/or flex as the dilator 1401 moves through the vessel 40, while the second material portion 1405, comprising the harder material, can apply the outward radial force and cause radial expansion of the vessel 40 and/or the sheath 701.

FIG. 15 illustrates additional embodiments of a dilator 1501 comprising the tapered end portion 713. In aspects, the second tapered region 727, the central region 725, and the proximal shaft region 729 of the dilator 1501 may be substantially identical to the second tapered region 727, the central region 725, and the proximal shaft region 729 of the dilator 703. However, in contrast to the first tapered region 723 of the dilator 703, the dilator 1501 may comprise a first tapered region 1503 that increases in diameter from the distal end 719 to the first central end 731 at a non-constant rate. For example, the first tapered region 1503 can comprise a first tapered portion 1505 and a second tapered portion 1507. The first tapered portion 1505 can extend a distance from the distal end 719 toward the first central end 731. The first tapered portion 1505 may be substantially identical to a corresponding region of the first tapered region 723 of the dilator 703, with the first tapered portion 1505 increasing in diameter at a constant rate from the distal end 719. However, the second tapered portion 1507 can be positioned between the first tapered portion 1505 and the central region 725, with the second tapered portion 1507 increasing in diameter at a non-constant rate from the first tapered portion 1505 to the central region 725. For example, the second tapered portion 1507 can comprise a valley portion 1509 that is rounded in an axial direction along the dilator axis 715. The second tapered portion 1507 can initially (e.g., from the first tapered portion 1505) increase in diameter at a gradual rate (e.g., along a first half of the second tapered portion 1507) followed by increasing in diameter at a faster rate (e.g., along a second half of the second tapered portion 1507). In this way, the first tapered region 1503 can comprise one portion (e.g., the first tapered portion 1505) that increases in diameter at a constant rate and another portion (e.g., the second tapered portion 1507) that increases in diameter at a non-constant rate.

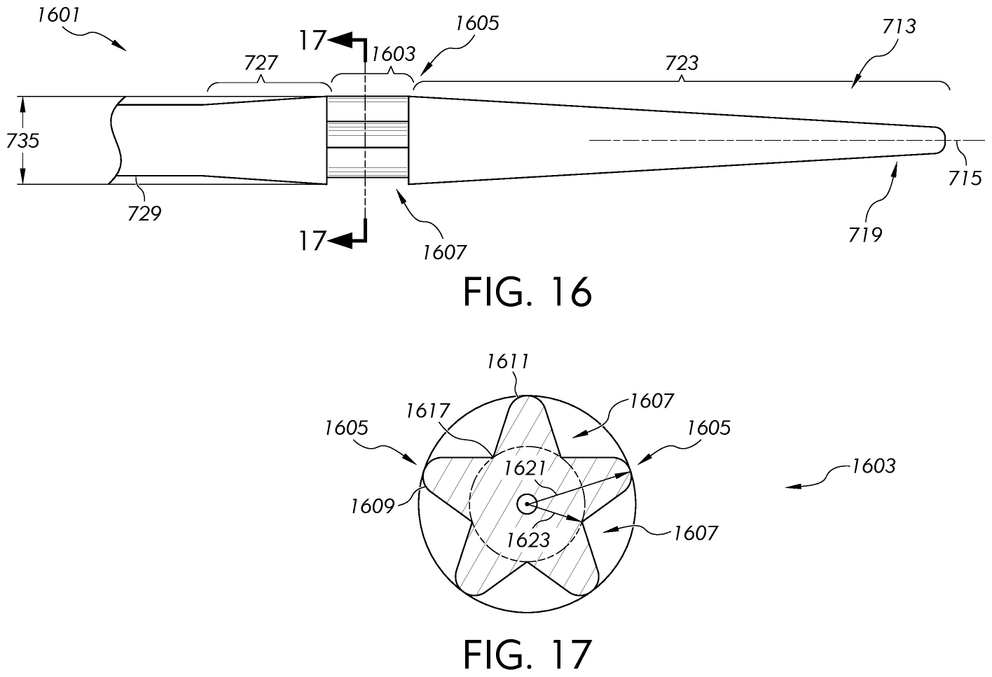

FIGS. 16-17 illustrate additional embodiments of a dilator 1601 comprising the tapered end portion 713. FIG. 16 illustrates a side view of the tapered end portion 713 of the dilator 1601 and FIG. 17 illustrates a cross-sectional view of FIG. 16 along lines 17-17 of FIG. 16. In aspects, the first tapered region 723, the second tapered region 727 and the proximal shaft region 729 of the dilator 1601 may be substantially identical to the first tapered region 723, the second tapered region 727 and the proximal shaft region 729 of the dilator 703. However, in contrast to the central region 725 of the dilator 703, the dilator 1601 may comprise a central region 1603 in which a cross-sectional size of the central region 1603 is non-constant about a circumferential perimeter of the central region 1603.

Referring to FIG. 17, the central region 1603 can comprise a plurality of peak portions 1605 and a plurality of valley portions 1607. The plurality of peak portions 1605 can comprise a first peak portion 1609, a second peak portion 1611, etc., and the plurality of valley portions 1607 can comprise a first valley portion 1617, etc. While the dilator 1601 in FIG. 17 is illustrated as comprising five peak portions and five valley portions, any number (e.g., one or more) of peak portions and/or valley portions may be provided. In aspects, the first valley portion 1617 may be located circumferentially between the first peak portion 1609 and the second peak portion 1611 about the circumferential perimeter of the central region 1603. In this way, the central region 1603 can comprise alternating peak portions and valley portions, with a valley portion positioned between two peak portions, and a peak portion positioned between two valley portions. The plurality of peak portions 1605 can comprise a peak radius 1621 and the plurality of valley portions 1607 can comprise a valley radius 1623. In aspects, the peak radius 1621 may be greater than the valley radius 1623, such that the cross-sectional size, or radius, of the central region 1603 may be non-constant about the circumferential perimeter. In aspects, the peak radius 1621 may be about half of the second diameter 735 such that the central region 1603 can comprise a cross-sectional size that is substantially equal to the second diameter 735 of the central region 725. In this way, the dilator 1601 can function substantially identically to the dilators 703, 1201, 1301, 1401, 1501 illustrated in FIGS. 7-15, with the central region 1603 causing radial-expansion of the vessel 40 and/or the sheath 701. Due to the central region 1603 comprising the non-constant cross-sectional size, the total radial force exerted upon the walls 705, 1101 about a perimeter of the central region 1603 may be reduced, such as, for example, by being limited to the plurality of peak portions 1605 that contact the walls 705, 1101.

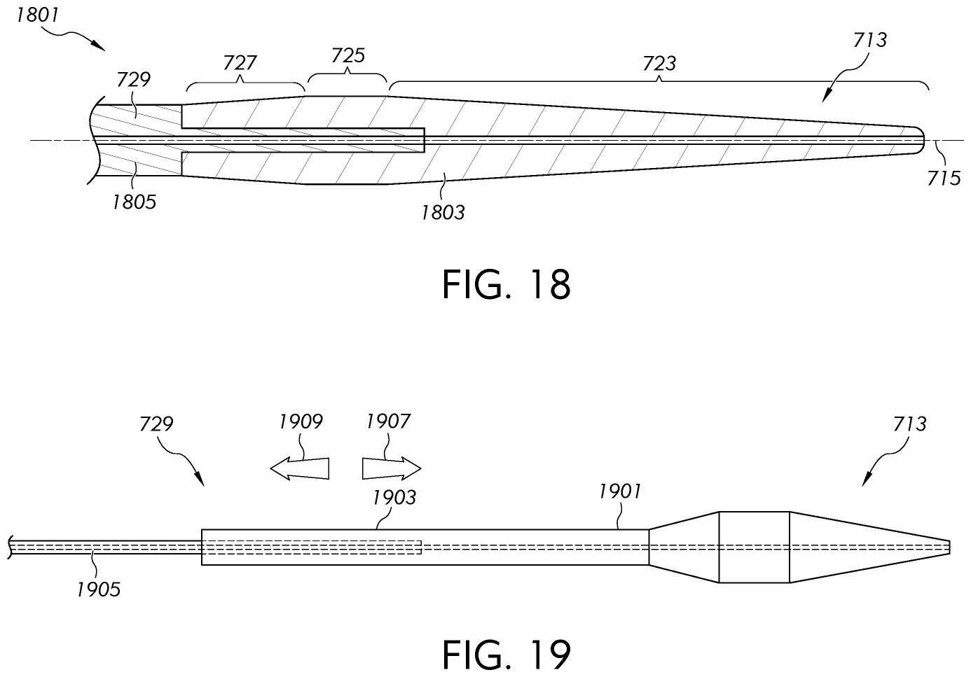

FIG. 18 illustrates additional embodiments of a dilator 1801 comprising the tapered end portion 713. In aspects, the first tapered region 723, the second tapered region 727, the central region 725, and the proximal shaft region 729 of the dilator 1801 may be substantially identical in shape and function to the first tapered region 723, the second tapered region 727, the central region 725, and the proximal shaft region 729 of the dilator 703. However, the dilator 1801 can comprise a plurality of materials, for example, with the first tapered region 723, the second tapered region 727, and the central region 725 comprising a material that is different than a material of the proximal shaft region 729. For example, the dilator 1801 can comprise a first material portion 1803 and a second material portion 1805. The first material portion 1803 can comprise the first tapered region 723, the second tapered region 727, and the central region 725, and the second material portion 1805 can comprise the proximal shaft region 729. In aspects, the second material portion 1805 can comprise a softer and more flexible material than the first material portion 1803. In this way, the second material portion 1805 can bend and/or flex as the dilator 1801 moves through the vessel 40, while the first material portion 1803, comprising the harder material, can apply the outward radial force and cause radial expansion of the vessel 40 and/or the sheath 701. In aspects, the first material portion 1803 can be formed by multi-material injection molding, for example, over-molding.

FIG. 19 illustrates additional embodiments of a dilator 1901 comprising the tapered end portion 713. In aspects, the first tapered region 723, the second tapered region 727, and the central region 725 of the dilator 1901 may be substantially identical to the first tapered region 723, the second tapered region 727 and the central region 725 of the dilator 703. However, the proximal shaft region 729 of the dilator 1901 can comprise a plurality of shaft portions, for example, a first shaft portion 1903 and a second shaft portion 1905. In aspects, the at least one lumen 771 can extend through the first shaft portion 1903 and the second shaft portion 1905. The first shaft portion 1903 can be attached to the second tapered region 727. The second shaft portion 1905 can be received at least partially within the first shaft portion 1903, for example, with a channel defined within the first shaft portion 1903. In this way, the first shaft portion 1903 and the second shaft portion 1905 can extend substantially coaxially along the dilator axis 715. However, in aspects, the first shaft portion 1903 can move relative to the second shaft portion 1905 in a first movement direction 1907 and/or in a second movement direction 1909. By moving in the first movement direction 1907, the first shaft portion 1903 can move away from the second shaft portion 1905 such that the dilator 1901 can elongate or increase in total length. The second shaft portion 1905 can remain within the channel of the first shaft portion 1903 such that the first shaft portion 1903 may not detach or separate from the second shaft portion 1905. By moving in the second movement direction 1909, the first shaft portion 1903 can move toward the second shaft portion 1905 such that the dilator 1901 can retract or decrease in total length. In this way, the length of the dilator 1901 can be adjusted, for example, by moving the first shaft portion 1903 relative to the second shaft portion 1905. The shaft portions 1903, 1905 can be implemented with none, some, or all of the dilators 703, 1201, 1301, 1401, 1501, 1601, 1801 disclosed herein.

It should be understood that while various aspects have been described in detail relative to certain illustrative and specific examples thereof, the present disclosure should not be considered limited to such, as numerous modifications and combinations of the disclosed features are possible without departing from the scope of the following claims.

What is claimed is:

1. A prosthetic valve delivery assembly comprising:
a dilator extending along a dilator axis between a proximal end and a distal end, the dilator comprising:
a first tapered region extending along the dilator axis between the distal end and a first central end, the first tapered region comprising a tapered shape with a first diameter at the distal end and a second diameter at the first central end, the second diameter greater than the first diameter;
a central region coaxial with the first tapered region and attached to the first central end, the central region comprising the second diameter that is substantially constant along a central length of the central region;
a second tapered region extending along the dilator axis between a second central end and a third central end, the second central end attached to the central region, the second tapered region comprising a tapered shape with the second diameter at the second central end and a third diameter at the third central end, the third diameter less than the second diameter; and
a proximal shaft region extending from and in contact with the third central end, the proximal shaft region comprising the third diameter such that a difference between the second diameter and the third diameter is within a French gauge range from about 3 Fr to about 5 Fr.

2. The prosthetic valve delivery assembly of claim 1, wherein the central region comprises a central length that is within a range from about 1 mm to about 150 mm.

3. The prosthetic valve delivery assembly of claim 1, wherein the tapered shape of the second tapered region comprises a taper angle that is within a range from about 1 degree to about 30 degrees.

4. The prosthetic valve delivery assembly of claim 1, wherein the second diameter is less than about 1.5 times a vessel diameter of the vessel.

5. The prosthetic valve delivery assembly of claim 1, further comprising a handle attached adjacent to the proximal end of the dilator, the handle axially fixed relative to the dilator and comprising a non-constant cross-sectional size along a length of the handle.

6. The prosthetic valve delivery assembly of claim 1, wherein the dilator comprises at least one lumen extending axially through the dilator.

7. The prosthetic valve delivery assembly of claim 6, wherein the at least one lumen comprises a first lumen, a second lumen, and a third lumen, the first lumen laterally offset from, and between, the second lumen and the third lumen.

8. The prosthetic valve delivery assembly of claim 1, wherein the dilator comprises a plurality of materials.

9. A prosthetic valve delivery assembly comprising:
a sheath comprising a wall surrounding an elongated chamber, the sheath configured to be received within a vessel; and
a dilator configured to be received within the chamber and extending along a dilator axis between a proximal end and a distal end, the dilator comprising:
a first tapered region extending along the dilator axis between the distal end and a first central end, the first tapered region comprising a tapered shape with a first diameter at the distal end and a second diameter at the first central end, the second diameter greater than the first diameter;
a central region coaxial with the first tapered region and attached to the first central end, the central region comprising the second diameter that is less than about 1.5 times a vessel diameter of the vessel, the central region comprising a central length that is within a range from about 1 mm to about 150 mm;
a second tapered region extending along the dilator axis between a second central end and a third central end, the second central end attached to the central region, the second tapered region comprising a tapered shape with the second diameter at the second central end and a third diameter at the third central end, the third diameter less than the second diameter, the tapered shape of the second tapered region comprising a taper angle that is within a range from about 1 degree to about 30 degrees; and
a proximal shaft region extending from and in contact with the third central end, the proximal shaft region comprising the third diameter, the third diameter within a range from about 60% to about 90% of the second diameter.

10. The prosthetic valve delivery assembly of claim 9, wherein a diameter of the central region is non-constant along the central length of the central region, and the second diameter is a maximum diameter of the central region.

11. The prosthetic valve delivery assembly of claim 10, wherein the central region comprises a first axial location and a second axial location comprising the second diameter, and a third axial location is positioned between the first axial location and the second axial location and comprises a diameter less than the second diameter.

12. The prosthetic valve delivery assembly of claim 9, wherein a cross-sectional size of the central region is non-constant about a circumferential perimeter of the central region.

13. The prosthetic valve delivery assembly of claim 9, wherein the second diameter is constant along the central length of the central region.

14. The prosthetic valve delivery assembly of claim 9, wherein the central region comprises a material that is different than a material of the proximal shaft region.

15. A method of expanding a sheath comprising:
positioning a sheath within a vessel, the sheath comprising a wall surrounding an elongated chamber;
inserting a dilator within the chamber, the dilator extending along a dilator axis between a proximal end and a distal end, the dilator comprising a first tapered region with an increasing diameter from the distal end, a central region coaxial with and in contact with the first tapered region and comprising a substantially constant diameter, and a second tapered region extending from and in contact with the central region, the second tapered region comprising a tapered shape with a decreasing diameter from the central region; and
radially expanding the sheath by contacting the wall with the central region.

16. The method of claim 15, wherein the central region comprises a central length that is within a range from about 1 mm to about 150 mm.

17. The method of claim 15, further comprising moving the dilator axially by applying a force to a handle that is attached adjacent to the proximal end of the dilator, the handle fixed relative to the dilator and comprising a non-constant cross-sectional size along a length of the handle.

18. The method of claim 15, further comprising receiving a first guidewire within a first lumen that extends axially through the dilator.

19. The method of claim 18, further comprising receiving a second guidewire within a second lumen that extends axially through the dilator, and a third guidewire within a third lumen that extends axially through the dilator, the first lumen laterally offset from, and between, the second lumen and the third lumen.

20. The method of claim 15, further comprising radially expanding the vessel by contacting a vessel wall of the vessel with the central region.
